(19) United States
(12) Patent Application Publication
(10) Pub. No.: US 2008/0219939 A1
(43) Pub. Date: Sep. 11, 2008

(54) Sunblock formulations

(76) Inventor: Guerry L. Grune, Virginia Beach, VA (US)

Correspondence Address: GUERRY LEONARD GRUNE, 784 S VILLIER CT., VIRGINIA BEACH, VA, 23452, US

(21) Appl. No.: 12/074,907

(22) Filed: Mar. 7, 2008

Related U.S. Application Data
(60) Provisional application No. 60/905,571, filed on Mar. 7, 2007.

(51) Int. Cl. A61K 8/92; A61Q 17/04

(52) U.S. Cl. 424/59

(57) ABSTRACT

Disclosed is a sunblock composition providing ultraviolet radiation protective agents that can be used in various compositions or formulations, specifically those of a higher SPF value that utilize micronized zinc dioxide, micronized titanium oxide, glass microspheres, glycerin and/or aloe, a carrier oil, and an emulsifier. The emulsifier is selected from the group comprising fatty acid salts, fatty acid sucrose esters, phospholipids, borate salts, cocoate esters, essential oils, non-petroleum derived waxes, chitosan, water and/or ethanol, a thickening agent, a pigment and a carotenoid or any single or multiple combination of this group of emulsifiers.

This application takes priority under 35 USC 119(e) of Provisional Application 60/905,571 filed 7 Mar. 2007 titled “Sunblock Formulations”.

### FIELD OF DISCLOSURE

This disclosure relates to ultraviolet radiation protective agents that can be used as beneficial sunscreens and sun-blocks in various compositions or formulations, specifically those of a higher SPF value (SPF 15-30, or greater).

### BACKGROUND OF THE DISCLOSURE

Although a tan has long been considered a symbol indicative of good health and the ability to secure sufficient leisure time to enjoy many and numerous outdoor activities, it has become very evident that excessive exposure of the human skin to sunlight is harmful.

Numerous sunblocking agents/sunscreens include compounds in the overall composition that are endocrine disrupters and therefore are toxic to mammalian cells by actually causing cells to die in the presence of both active and inactive agents included in the compositions. It would be advantageous to provide sunblock formulations that reduce or eliminate the presence of endocrine disrupters.

### SUMMARY OF THE INVENTION

Ultraviolet radiation protective agents, in combination with lotions, cremes, pastes, sprays, lip balms, cosmetics, etc., are disclosed. The agents can be used as beneficial sunscreens, for example, as sunscreens, in various compositions or formulations. The compositions include cytoprotective additives, which can provide the skin of the user with enhanced protection and increased immuno-responsiveness, relative to conventional sunscreen formulations. As used herein, the term “sunscreen” includes both sunscreens and sunblocks.

Ideally, the formulations do not include substantial amounts of endocrine disruptive agents. The term “substantial amounts,” means more than 5% by volume, or not enough endocrine disruptive agents to drastically kill cells on contact as defined by the LUMI-CELL™ MER Bioassay. In some embodiments, the formulations enhance the skin's immuno-responsiveness from cancerous or pre-cancerous skin cells in the presence of radiation, such as UV light or sunlight. In one embodiment, the formulation includes greater than 90%, ideally greater than 95%, and more ideally, greater than 98% by volume agents that will not kill cells and primarily all earth-grown ingredients, while still providing an SPF value of at least 15 or greater. In some aspects, the sunblock is translucent or transparent upon application to human skin, although at higher SPF levels, where higher concentrations of pigments such as zinc and titanium dioxide are used, translucency may or may not be completely achievable. Where translucency is not achievable, the formulation can include metal oxides and other pigments that assist with matching the skin tone and color of the user.

The formulations include inorganic sun-block agents and/or known non-toxic sunscreen agents, such as zinc oxide and/or titanium dioxide. When zinc oxide and/or titanium dioxide are used, they are preferably present in micronized form (i.e., micrometer or nanometer particle size ranges), and in preferred amounts and ratios as described herein.

The formulations also include aloe, preferably cold-pressed aloe. The aloe includes an oligosaccharide with a molecular weight of approximately 1-5,000 Daltons that is glucose rich, and also contains mannose. The oligosaccharide inhibits the loss of skin immunocompetency. Cold-processed aloe that is processed within 45 minutes of harvesting contains about 200 biologically active agents, and the effect of all of these agents is desirable to further enhance the cytoprotective ability inherent in aloe plant extracts.

While not wishing to be bound by a particular theory, the use of aloe in the sunscreen formulation permits one to use relatively lower amounts of the sunscreen active ingredients than in conventional sunscreen formulations, while still achieving relatively high SPF values, and without the unsightly whiteness which occurs in prior sunscreen compositions at concentrations specifically TiO₂ and/or ZnO (even micronized TiO₂) above about 5%. The instant sunscreen formulations can use relatively higher concentrations of micronized zinc oxide and/or titanium dioxide without incurring a whitening effect, e.g., even up to 25% each, with acceptable appearance—meaning little or no whitening effect on the skin. When the titanium dioxide and zinc oxide are present in the form of micronized particles, in ratios of from about 1:4 to 1:3.5 to 1:3.3, the formulations have been found to leave little or no unsightly white residue when applied to the skin.

While hydrophilic titanium dioxide preparations can be used, they are not necessary. Further, energy intensive processes such as powder milling are not required, nor are organic active sunscreens required for high efficacy. The formulation can be blended using homogenizers to mix the components.

The formulations described herein include at least the following components, and can be formulated into pastes, lotions, sprays, lip balm, crèmes, and other cosmetic or make-up products:
an inorganic sun-block agent including any metal oxide, glass micropheres, silica and silica compounds, and optionally metal oxide pigments with particles that are micronized, nanosized, or otherwise,
an emulsifier or mixtures thereof;
an oil component comprising a carrier oil, preferably an essential oil;
at least one emollient, where the emollient may be the emulsifier of (b) above sun protective factor (SPF) boosting additives sufficient for imparting an SPF value of 15 or greater
texturing agents providing a powdery feel,
optionally carotenoids or other effective antioxidants which are translucent or transparent with respect to the present compositions,
optionally inorganic pigments to provide color and texture matching of the skin.

The emollient or emulsifier is preferably aloe as it is “cold pressed” or an extract of aloe that is currently removed during normal processing and recovered by some means. The aloe or its extract may not provide sufficient emulsification with regard to the remaining ingredients of the composition. It has since been determined that a single species of aloe is preferred and best for providing a healthy and well dispersed product with the highest known concentrations of cytoprotective agents—aloe barbadensis Miller-Stockton. Aloe is available in gel and in oil forms. It may also be possible to use a liposome or phospholipid such as Phosphatidyl-choline (PC), as described below.

An emulsifier (also known as a surfactant from surface active material or emulgent) is a substance which stabilizes an emulsion. An emulsion is a mixture of two immiscible (unblendable) substances. One substance (the dispersed phase) is dispersed in the other (the continuous phase). Examples of emulsifiers are egg yolk (where the main emulsifying chemical is the liposome or phospholipid lecithin), and mustard, where a variety of chemicals in the mucilage surrounding the seed hull act as emulsifiers; proteins and low-molecular weight emulsifiers are common as well. Whether an emulsion turns into a water-in-oil emulsion or an oil-in-water emulsion depends of the volume fraction of both phases and on the type of emulsifier. Generally, the Bancroft rule applies: emulsifiers and emulsifying particles tend to promote dispersion of the phase in which they do not dissolve very well; for example, proteins dissolve better in water than in oil and so tend to form oil-in-water emulsions (that is they promote the dispersion of oil droplets throughout a continuous phase of water).

Emollients are substances which soften and soothe the skin. They are used to correct dryness and scaling of the skin. The terms ‘moisturizer’ (to add moisture) and ‘emollient’ (to soften) are sometimes used interchangeably as they describe different effects of these agents on the skin. However, the term emollient is most often used to describe single ingredients, whereas ‘moisturizer’ describes finished products. Emollients have three basic actions: 1) Occlusion—providing a layer of oil on the surface of the skin to slow water loss and thus increase the moisture content of the stratum corneum. 2) Humectant—increasing the water-holding capacity of the stratum corneum. 3) Lubrication—adding slip or glide across the skin. An example of an emollient that will boost the occlusivity of the present disclosure is chitosan. Chitosan is a linear polysaccharide composed of randomly distributed β-(1-4)-linked D-glucosamine (deacetylated unit) and N-acetyl-D-glucosamine (acetylated unit). Chitosan is produced commercially by deacetylation of chitin (can be produced from chitin also), which is the structural element in the exoskeleton of crustaceans (crabs, shrimp, etc.). Chitosan enhances the transport of polar drugs across epithelial surfaces, and is biocompatible and biodegradable. Purified qualities of chitosans are available for biomedical applications. Chitosan and its derivatives such as trimethylchitosan (where the amino group has been trimethylated) have been used in non-viral gene delivery. Trimethylchitosan, or quaternised chitosan, has been shown to transfect breast cancer cells. As the degree of trimethylation increases the cytotoxicity of the derivative increases. At approximately 50% trimethylation the derivative is the most efficient at gene delivery. Oligomeric derivatives are relatively non-toxic and have good gene delivery properties. Aloe acts as an emollient in the present compositions as well.

The compositions of this disclosure provide formulations having an SPF of at least 10, with titanium dioxide, zinc oxide, or a combination of the two (with or without silica or silicon dioxide and/or cosmetic glass microspheres, or other metal oxides including but not limited to magnesium, manganese, iron, copper, etc.), with a treated or untreated hydrophilic surface, at concentration levels of at least 4% and preferably at least 20% to reach SPF 15 or greater. The compositions of this disclosure exhibit extremely efficient uses of sunblocking components, particularly zinc oxide. Alternatively, higher levels of preferably micronized titanium dioxide or zinc oxide can be used with or without ultramarine pigments added to the composition. These pigments are known to eliminate the whiteness and poor spreadability of currently available compositions. The sun-block agent inorganic/organic dispersion can be made in the following way:

Essentially, the compositions of this disclosure are easily made by homogenization (high speed/shear mixing) and provide an excellent dispersion of the inorganic based sunscreen/sunblock agent throughout the composition, thus ensuring even skin coverage. With or without the use of ultramarine pigments, the compositions are substantially invisible upon application to the skin.

Thus, in one possible embodiment, the present disclosure is directed toward a colored sunscreen emulsion comprising:
at least one ultramarine pigment that imparts a color other than white to the emulsion with a titanium dioxide or zinc oxide or possibly fumed or fused silica or even silicon dioxide or micronized glass cosmetic spheres so that when the emulsion is rubbed into the skin, the color substantially disappears;
at least one sunscreen active agent in an amount effective to protect skin against the actinic radiation of the sun—this preferably being ZnO or Z-Cote® (micronized particles—preferably nanoparticle sized to assure transparency);
a cytoprotective substance such as a glucose-rich mannose-containing oligosaccharide obtained from and used with aloe barbadensis Miller as the at least one emulsifier; and
sufficient water to form the other than a white colored emulsion; and sufficient dispersion to assure SPF of at least 15 and an SPF booster that shows no appreciable toxicity.

The amount of the ultramarine pigment in the composition can range from 0 to about 25 weight percent of the composition, and preferably from about 1 to about 5 weight percent of the final formulation.

Optionally, the present compositions can contain one or more additional ingredients, including emollients, waterproofing agents, dry-feel/powdery-feel modifiers, insect repellants, antimicrobial preservatives and/or fragrances.

In another embodiment, the present disclosure is directed towards a method for protecting the skin against sunburn while increasing mammalian skin cell immuno-response to cancerous skin cells while eliminating possible endocrine disruption response of human organs comprising topically applying the sunscreen formulation, as described above, to the skin.

An advantage of the present disclosure is that it provides a sunscreen and a method for protecting against sunburn that enables the user to apply the sunscreen more completely and uniformly to the skin, thus providing more effective protection against skin damage and homogenously enhancing cytoprotection while eliminating endocrine disruptive organics, thus providing for long term health and safety in the presence of UV light.

Another advantage of the present disclosure is that it provides a sunscreen with a color indicator which has a low fabric staining potential, and for which those stains that form can easily be removed from fabrics.

Still yet another advantage of the present disclosure is that it provides an optionally colored sunscreen and a method for protecting against sunburn that is more enjoyable for human use because of the attractiveness and appealing nature of the color indicator. For domesticated animals as well as humans, the use of matching colors may also be appealing.

This disclosure allows for the use of ultrafine ZnO particles that are invisible when applied to human skin—this is normally less than 0.5 microns and most often at less than 0.2 microns. This “invisible” ZnO would be the primary and perhaps only sunblock “active” ingredient or could be combined with titanium dioxide and silica or silicon dioxide and cosmetic microspheres and optionally inorganic pigments to enhance dispersion when mixed with homogenization equipment and therefore provide a higher SPF value. Metal oxide pigments are also useful and included as part of this invention.

### DETAILED DESCRIPTION

The UV-protective compositions of this disclosure yield highly effective ultraviolet (UV) blocking capabilities. A typical titanium dioxide sunscreen composition of SPF 15 requires levels of titanium dioxide that impart a significant whitening effect to the skin; the compositions of this disclosure minimize this disadvantage and are therefore also economically viable to produce. Use of micronized titanium dioxide which is submicron sized has also been used in formulating the compositions of the present disclosure.

The composition of this disclosure include emulsions that are cosmetically superior to conventional inorganic preparations, including water-in-oil titanium dioxide-only formulations, at equivalent SPF ratings, due to the method and type of dispersion as described above. The compositions of this disclosure can be used for sun protection in daily wear or facial products as well as for recreational situations. Because of the efficiency of the system, the inventive formulations are significantly better than the prior art in that they do not provide for additional toxic or cell killing agents to be included in either the active or inactive list of ingredients. There are several ingredients that contribute to the unexpectedly high efficiency of the compositions blocking of UV radiation. It has been found, however, that only one known organic UVA protector, butyl-methoxydibenzoylmethane has been shown to be benign regarding activity in cells or developmental effects on animals. Depending on the need for individual formulations based on the inventive concept herewithin, the use of this or other UVA protectors may be required. The formulations of this disclosure is intended to filter harmful UVA as well as harmful UVB radiation so that the skin is fully protected, but primarily are designed to block light.

Ease of application and cosmetic appeal, on the other hand, are important in formulating sunscreen compositions. These characteristics rely on subjective evaluations such as visual and tactile impressions by the user. Consumer research studies indicate that a sunscreen formulation should rub in easily, leave the skin non-sticky and, above all should be invisible or at least translucent to match skin color of the skin after application. Sunscreen compositions containing organic sunscreen agents have been found, in some cases, to irritate the skin. A recent development in the reduction of particle sizes of ZnO has resulted in microfine essentially clear ZnO when applied to the skin. Formulation in the family known as Z-Cote® which is a trademarked composition sold by BASF is one such example of a micronized zinc oxide available today. (The process of micronization refers to breaking up a substance into particles that are only a few micrometers in substance—or submicrometer in size.) The groups of inorganic sun-block agents includes titanium dioxide, micronized titanium dioxide, zinc oxide, micronized zinc oxide, iron oxide, silicon dioxide, magnesium oxide, manganese oxide, silica, alumina, and aluminum oxides and other metal compounds that can provide safe and effective sunblocking compositions such as those using Boron. Cosmetic microspheres, such as those made of silica dioxide or silica such as CM-111 AS produced by 3M Corp of St. Paul, Minn., can also be used as an inorganic sun-block agent.

In addition, the need for an acceptable emollient that reduces the negative affects associated with abrasive inorganics and that also includes the benefit of providing cytoprotection and healing of the skin is necessary. Allowing for the reduction of irritation or sensitization of the skin suggests that “cold-pressed” Aloe and Chitosan are useful ingredients for such a UV-protective formulation.

It has also been determined that it is quite difficult, if not impossible, for current dispersion systems of micronized TiO₂, ZnO, SiO₂ and the like to be non-toxic. As presented in earlier disclosures, the Lumi-cell test technique (developed by Xenobiotics Laboratories of Durham, N.C.) can be used to determine if cells are killed. Therefore, in essence, using one of several definitions of toxicity—adverse effects occurring as a result of repeated daily dosing of a chemical or exposure to the chemical, for part of an organism's lifespan—the dispersions themselves are toxic. The present disclosure includes the possible use of aloe, not only as an emollient, but also as a very effective dispersing agent for the inorganic micronized (and larger) sunblock active agents. High speed shearing (accomplished with the use of an homogenizer), followed by high speed mixing (up to 40,000 rpm with an homogenizer) provides a consistent, usable, and easily blendable inorganic/organic dispersion free of any known toxic substances (if the aloe source and inorganic particle source is well documented and controlled). A well mixed and homogeneous dispersion is essential in providing sufficient homogeneity and SPF values with any associated non-active cream, lotion, gel, spray, etc. that is used to provide a formulation consistent with the basis of the present disclosure. It has been found recently that the use of an homogenizer is essential to prevent separation of (primarily oil-based) components during extended shelf-life and that there are synergistic effects both in terms of transparency and SPF boosting capabilities in the final compositions that require homogenization.

### Non-Toxic SPF Boosting Agents

To provide the proper SPF value, it may be desired to enhance or boost the SPF number using boosting agents. These boosting agents should also not be toxic (cell-killing). It is likely that many natural oils and perhaps derivatives of other natural occurring substances (such as essential oils of safflower, sunflower, rice bran, eucalyptus, rosemary, peru balsam, olibanum, orange, almond, sesame, ylang ylang, jojoba, or coconut) that can provide dispersion capabilities to enhance or boost SPF values may also be determined to be toxic free and are therefore also part of this disclosure. It has also been suggested that to increase SPF values for both in vivo and in vitro testing, film forming properties are important. The following film forming agents may also be used in the formulations and resulting compositions of the present disclosure: wheat protein extract, silk protein, galactoarabian (aribinogalactan), marine collagen, pea extract, purcellin oil, preen oil, and wild mango butter.

Bentonite can be used to boost SPF values. Colloidal Bentonite contains the active constituent montmorillonite super-refined with demineralized water as a vehicle. The liquid bentonite was the first of its kind to be processed removing the dirt, mica and impurities leaving the active ingredient Montmorillonite in a colloidal suspension. The Montmorillonite molecule has a shape similar to a business card with the wide surfaces negative and the edges of the card positively charged. This allows it to have many times more negative than positive charges. In addition, the very minuteness of the particles of Montmorillonite provides a large surface area in proportion to the volume used, thus enabling it to pick up many times its own weight in positively charged particles. To obtain maximum effectiveness in the human body, it must be put in a liquid colloidal-gel state. When a volcano erupts, there is often a fine steam or mist released which contains a substance known as volcanic ash. Bentonite is a volcanic ash. As it contains many minerals (24 to 33), it serves to mineralize the soil. Bentonite clay can be mined from veins which are two to three feet wide and deep, but many yards long. Natives on every continent have used volcanic ash for centuries both internally and externally. The value of montmorillonite (the active ingredient in bentonite) lies in its ability to adsorb (not absorb) many times its own weight and volume in an aqueous medium. It has a predominantly negative charge that is capable of attracting many kinds of positively charged particles. Its negative charge enables it to pick up positively charged, toxic material from the alimentary canal to be expelled in the feces. The adsorption is a rapid process and can quickly neutralize allergens before they attach themselves to blood cells, thus preventing allergic reaction.

Aloe Vera gel serves numerous purposes in the present disclosure, including acting as a dispersant, as an emollient, boosting the SPF value, and improving aesthetics, and is believed by many to have healthful benefits. For medicinal purposes, aloe vera is most commonly used externally to treat various skin conditions, and burns. Not only does it soothe the skin, ease pain and reduce inflammation, studies have been done to show that using aloe as a topical treatment for burns will help speed up the healing recovery process. Many cosmetic companies are now adding this plant to products including makeup, soaps, sunscreens, shampoos and lotions, as well as any product that is created to soothe, protect and moisturize the skin. This is due partially to the fact that aloe extract is full of vitamins, nutrients and minerals.

One embodiment of the present disclosure includes the use of a pure strain of aloe-vera known as aloe barbadensis Miller-Stockton. This strain or species of aloe is believed to have low concentrations of the enzyme aloin. Aloin is an enzyme which when taken internally has a diuretic effect (i.e. it causes diarrhea) by causing inflammation within the human intestinal tract. The Stockton strain is believed to be low in aloin because the product is marketed for internal consumption and has not had any documented diuretic effect on thousands of users over the course of more than 30 years. Further, the Stockton strain is believed to include a greater concentration of cytoprotective oligosaccharides. Utilizing the Stockton strain of aloe for the formulations of the present disclosure ensures purity, uniformity, and a proper medium for dispersing the active inorganic sun-block agents. Further, the Stockton strain is ideal because it is a single species source and therefore reproducible on a batch-to-batch basis. The Stockton strain is not mixed with any other strains of aloe which are known to possess large doses of aloin or other impurities including toxic and even poisonous constituents if consumed. Further, the Stockton strain is ‘cold-pressed’ mechanically and not processed chemically by carbon adsorption or any other chemical means. The aloe processing industry includes carbon adsorption to prevent color loss. However, the carbon adsorption process also removes some or all of the cytoprotective oligosaccharides which the present disclosure requires. By using a single species of aloe, it is also possible to maximize the most advantageous health features of the plant (minimize any unhealthy features) used in any of the compositions of the present disclosure.

### Cytoprotective Oligosaccharides

In one embodiment, the sunblock includes cytoprotective oligosaccharides from aloe barbadensis Miller. These oligosaccharides prevent preventing damage to the skin immune system caused by harmful UV radiation. “Cold-pressed” Aloe which contains the beneficial oligosaccharides and provides an emollient base for the UV protective formulation is possibly the best known choice as a cytoprotective agent that inhibits the loss of skin immuno-competency induced by ultraviolet radiation, as this agent is readily available and comparably inexpensive. Other such inhibitors include amino-acids, vitamins or pro-vitamins, nucleo-derivatives, and vegetable extracts. Amino acids comprise tryptophan, histidine, phenylalanine, tyrosine, said vitamins and provitamins comprise vitamin B6, vitamin A, vitamin E, tocopherols and in particular D-alpha tocopherol, beta carotene, bioflavonoids, nucleotides and polymers thereof, cascara, frangula, camomile, hyperic, calendula, elicriso, licorice or essential oils thereof all may have similar cytoprotective or immune boosting effects on mammalian skin. The essential oils of frankincense and rosemary have been found to work effectively and synergistically in strengthening the neuromuscular response of patients who are exposed to its scent in combination with compositions of the present disclosure. For the purposes of clarifying issues due to the use of essentially any free radical scavenger and/or anti-oxidant compound that is beneficial to human health in combination with the formulations and compositions of the present disclosure, it is known that such compounds are also cytoprotective agents.

### Enhanced Immunoresponsiveness

In one embodiment, the sunblock includes compounds which provide enhanced immuno-responsiveness to the user. Representative compounds include extracts of aloe or similar naturally occurring substances (including kukua nut extract and similar anti-inflammatory agents that are naturally occurring substances).

Such substances would preferably not be processed, but if the beneficial anti-inflammatory effects are not lost during processing, then either the processed or non-processed substance may be used. The importance of processing within a short time period after harvesting the aloe plant or other plants/nuts, etc. as well as keeping the plant and subsequent plant extract cool (at or below room temperature) during processing is now well understood. Essential oils including specifically frankincense and rosemary have been shown to have immuno-enhancing properties, as determined by Kinesiologist Dr. John Schmidt of Triangle Wellness Center at 182 Wind Chime Ct. Ste. 203 Raleigh, N.C. 27615. This was determined by a strengthening in neuromuscular response using scent (aroma or aromatherapy) testing of these essential oils. The testing was performed using oils together with compositions of the present disclosure and using the oils either alone or in combination.

Carotenoids are powerful antioxidants found in abundance in certain fruits and vegetables. They neutralize damaging free radical molecules and are absorbed in human plasma and tissue, providing an excellent indicator of a person's antioxidant level. Carotenoids have many physiological functions. Given their structure, carotenoids are efficient free-radical scavengers, and they enhance the vertebrate immune system. Consequently, epidemiological studies have shown that people with high beta-carotene intake and high plasma levels of beta-carotene have a significantly reduced risk of lung cancer.

Agents which Absorb or Quench Free Radicals or Boost Carotenoid Levels

With overexposure to UV light, the human immune system becomes depressed. Most sunscreens further compromise the immune system by including ingredients which lead to the creation of additional free radicals—organic sunscreens that decompose in the presence of UV light. The formulations of the present disclosure, by contrast, contain ingredients which absorb or quench free radicals so that they can not further damage the cells of the skin. The compositions contains ingredients known to boost carotenoid levels, including cold-pressed aloe vera gel and beta glucan, as well as Vitamins C & E, which bind with carotenoids to further boost anti-oxidant levels in the skin.

Carotenoids are a family of natural fat-soluble nutrients important for antioxidant defense (Packer, 1992, 1993; Cadenas and Packer, 2002) found throughout the plant kingdom. They are responsible for the red, orange or yellow color of many fruits and vegetables, such as pineapples, citrus fruits, peaches, nectarines, persimmons, tomatoes, papaya, apricots, carrots, watermelons, pumpkins, squashes and sweet potatoes. Sometimes their presence is masked by chlorophyll, especially in dark green leafy vegetables like spinach, broccoli, collard greens, and kale. These substances also impart color to many birds (flamingo, ibis, canary, the Egyptian vulture's brightly colored yellow head), insects (lady bug), marine animals (crustaceans, salmon) and flowers.

More than 600 carotenoids have been identified in nature but less than 50 are abundant in the human diet. Among these, five carotenoids, β-carotene, a-carotene, lycopene, lutein, and zeaxanthin are found in the blood and known to be important in human health (Khachik et al., 1992; Gerster, 1993). A large number of epidemiological and experimental studies offer strong evidence that carotenoids are nutritionally important for normal cell regeneration (Clinton and Giovannucci, 1998; Clinton, 1999), eye health (Landrum et al., 1997; Cooper et al. 1999), plus numerous other health aspects linked to unstable oxygen molecules called free radicals (Rao and Agarwal, 2000; Cadenas and Packer, 2002).

### Anionic Emulsifiers

The formulations can include one or more of anionic emulsifiers, ideally emulsifiers derived from natural sources. In one embodiment, the emulsifier is a salt of a fatty acid, preferably a saturated fatty acid and/or straight-chain fatty acid. The fatty acid salt can be added to the formulation as the salts, or can be formed in situ. The salts can be alkali metal salts, alkali earth metal salts, or amine salts. Stearic acid salts, such as sodium stearate, are one example of a preferred emulsifiers, whereas isostearate salts tends to produce a composition which is not very efficient in the use of sunscreen, and oleate salts are unsaturated and tend not to result in efficient sunscreen compositions. While not wishing to be bound to a particular theory, it is believed that salts of straight-chain fatty acids work better than branched fatty acids, due to their relatively high melting point, above 70° C. or higher).

Sodium borate is another representative anionic emulsifier.

The anionic emulsifiers are typically present in amounts ranging from about 0.01 to about 10%, more preferably 0.1 to about 7% and most preferably from about 0.5 to about 5%.

Additional emulsifiers can be present in the formulations described herein, however, the formulations include at least one anionic emulsifier.

Phospholipids are another example of suitable anionic emulsifiers. Phosphatidylcholine (PC), also known as lecithin or PhosChol, is a representative phosopholipid. It is the major component of a phosphatide fraction which may be isolated from either egg yolk or soy beans, from which it is mechanically or chemically extracted using hexane. It is commercially available in high purity as a food supplement and for medical uses. It is regarded as a well tolerated and non-toxic surfactant/emulsifier, and is approved by the United States Food and Drug Administration for human consumption with the status “Generally Recognized as Safe”. Lecithin is an integral part of cell membranes, and can be totally metabolized, so it is virtually non-toxic to humans, whereas certain other emulsifiers can only be excreted via the kidneys. Some commercially available PC products are Phospholipon 90G® and Phospholipon 85G®, distributed by the American Lecithin Company of Oxford Conn. PC can be dispersed into an oil, glycerin, aloe vera, or otherwise suitable solvent before being added into the formulations described herein.

### Liposomes

The phospholipids described herein can be present in the form of liposomes. A liposome is a spherical vesicle with a membrane composed of a phospholipid bilayer used to deliver drugs or genetic material into a cell. Liposomes can be composed of naturally-derived phospholipids with mixed lipid chains (like egg phosphatidyl-ethanolamine), or of pure components like DOPE (dioleolylphosphatidylethanolamine). The lipid bilayer can fuse with other bilayers (e.g., the cell membrane), thus delivering the liposome contents.

Liposomes can be formed, for example, by sonicating phospholipids in water. Low shear rates create multilamellar liposomes, which have many layers like an onion. Continued high-shear sonication tends to form smaller unilamellar liposomes. In this technique, the liposome contents are the same as the contents of the aqueous phase. Liposomes can be used as emulsifiers in the same manner as the phospholipids discussed above.

### Humectants

The main purpose of any cream is to keep the skin moist. Many conventional creams form a suffocating film on the skin to prevent moisture loss. Humectants, such as the natural humectant, glycerin, actually attract water from the air and surrounding tissue. They keep the skin moist as long as there is sufficient moisture in the air. In a dry climate they actually draw moisture from the skin.

The formulations ideally include one or more humectants. Representative humectants include glycerine, collagen, elastin, panthenol (pro-vitamin B5) and keratin. Another representative humectant is Pepha®-Nutrix, a product of Pentapharm Ltd of Basel, Switzerland. Natural phospholipids, such as lecithin, can also be used. An important benefit of phospholipids is that they are hygroscopic (attract water from the surrounding air) and hold water where an increased level of hydration is needed. Therefore, phospholipids increase the hydration levels of the skin without being occlusive (forming a film to prevent water loss, and preventing normal cellular function).

### Agents to Promote a “Powdery Feel”

The formulation can include various agents to enhance the “powdery feel”, such as tapioca, sodium borate, glass microspheres, powdered milk or butter milk, sucrose stearate or sodium stearate, wood powders, and clays (such as bentonite). The amount needed to ensure the powdery texture together with the proper viscosity will vary from formulation to formulation, depending, among other things, on mixing sequence, homogenization of various oil/water/aloe components, temperatures, etc.

### Carrier Oils

The formulations can include a carrier oil. There are a range of different carrier oils each with their own individual properties and suitability for aromatherapy. The carrier oil may be selected from the group of essential oils or other known non-toxic esters. Other carriers include castor oil, avocado oil, broccoli seed oil, as well as keratin, and micronized or colloidal bentonite or the like which, although not oils have carrier capability.

Preferably, the carrier oil can be an essential oil, may be used together with some of the SPF boosting agents individually or in combination and should be present in the composition in an amount of between about 0.1% and about 20%. More preferably, it should be present in the amount of between about 1% and about 5%. Most preferably, it should be present in the amount of between about 2% and about 4%. Examples of essential oils include oils of jojoba, rice bran, sesame, safflower, almond, sweet almond, eucalyptus, sunflower, peru balsam, rosemary, olibanum, orange, sunflower, ylang ylang, apricot kernel, avocado, borage, cocoa butter, evening primrose, grapeseed, hazelnut, kukui, macadamia nut, olive, peanut, pecan, rose hip, bergamot, jasmine, neroli, patchouli, petitgrain, rose, vetiver, chamomile, mandarin, lavender, grapefruit, cypress, bay laurel, frankincense, clary sage, ginger, helichrysum, lemon, sandalwood, basil, black pepper, peppermint, geranium, wintergreen, thyme, tea tree, tangerine, spearmint, common sage, rosewood, pine, patchouli, oregano, nutmeg, myrrh, melaleuca, marjoram, manuka, lemon grass, lavender, juniper, ginger, cumin, clove, camphor, bay leaf, anise, allspice, and hyssop.

A number of the above mentioned essential oils, including jojoba and avocado, can be utilized in the present formulations also as emollients.

### Oil Phase Comprising the Carrier Oil and an Emollient

For conventional UV-protection formulations, an oil phase should contain at least two materials, the carrier oil or essential oil and a conventional emollient known to those of ordinary skill in the art as useful in sunscreen products, such as mineral oils, ester oils, vegetable oils, emollients such as fatty acid esters and the like. For the present disclosure, the use of a cold pressed aloe barbadensis Miller and specifically the Stockton species is to be substituted as an emollient or can be used in combination with the oils or emollients that are proven to be non-endocrine disrupting as well as not interfering with augmenting the cytoprotective enhancing effects of the known effective oligosaccharide aloe extract. The emollient should be present in the formulation in a ratio to the carrier concentration of from about 1:1 to about 3:1, most preferably, about 2:1. The carrier oil and the emollient should comprise from about 2% to about 40% of the total composition weight.

### Inorganic Sunscreens

A third major component which should be present in the compositions of this disclosure includes an inorganic sunscreen compound, such as titanium dioxide, zinc oxide or combinations thereof. Possible other inorganics include the use of fused or fumed silica or even silicon dioxide. Preferably, titanium dioxide, zinc oxide, silica, silicon dioxide, and/or cosmetic microspheres should be used having a primary particle size of less than about 300 nm in diameter. Larger particle sizes may also be used if necessary along with special pigments to ensure transparency or at least skin color matching. The inorganics should be present in the composition in the amount of from about 2% to about 25%. More preferably, it should be present in the amount of from about 2% to about 20%. The inorganic sunscreen compound should be oil dispersible, and may be present with or without surface coating.

The ratio of titanium dioxide or zinc oxide to the weight of the carrier oil and the emollient combined should be from about 0.0:1 to about 1:1. More preferably, the ratio should be between about 0.25:1 or 2:3, and most preferably 0.33:1.

In the case where salts of fatty acids are used care should be taken to keep the pH of the compositions of this disclosure at a level above about 5, more preferably, above about 5.5. Maintaining the pH at this level will ensure that these anionic emulsifiers remain in the salt form, which is important in retaining the stability and efficacy of the composition.

### Optional Components

The typical excipients present in a sunscreen emulsion system, such as a polymeric thickener/stabilizer, one or more additional emollient oils, microbial preservatives, waterproofing agents, antioxidants, fragrance, humectants, and water can also be present. Ideally, such excipients are selected such that they provide a non-toxic composition, and ideally improve, or do not reduce, the immunoenhancing and cytoprotective properties of other components in the formulation.

### Base Formulations

Base formulations of this disclosure may also be used as carrier compositions for active topical agents having dermatological effects, including depigmentation agents, anti-aging ingredients, anti-fungal agents, anti-microbial agents, and the like. For example, depigmentation agents can include magnesium ascorbyl phosphate or hydroquinone. Anti-aging agents can include retinoid compounds and alpha-hydroxy acids. Anti-fungal agents that can be included in the compositions of this disclosure include azole compounds including ketoconazole and the like. Anti-microbial agents may include triclosan.

The formulations and compositions described herein have a multi-action capability, as they would contain both sunscreen agents and other actives for protecting, treating, and enhancing the immuno-responsive nature of the skin, providing anti-aging properties, and adding cytoprotectiveness.

The compositions of this disclosure can be incorporated into various cosmetic and personal care products such as hand and body lotions, oils, ointments, lip balm products, facial cosmetics and the like.

One of the major challenges in providing the composition of the present disclosure is to provide a non-toxic, immuno-enhancing high (15 or greater) SPF formulation that can be readily achieved in a manufacturing environment for a reasonable cost. The use of aloe as both an emollient and a surfactant/dispersion agent together with either ZnO, titanium dioxide, silicon dioxide, fluoropolymers, silica, magnesium, glass microspheres, clays, wood powders, etc., micronized or otherwise (inorganic or acceptable organic sun-block agents) with optional additions of anti-oxidants such as caretonoids and dispersing agents including phosphotidyl choline or phospholipids, or liposomes or keratins in the manner detailed above and claimed has not been heretofore accomplished. The addition of SPF boosting agents that are not toxic is also unique to this disclosure and has heretofore not been previously considered or explored. It should be emphasized that SPF values of 15 or greater can be achieved solely by blending and subsequent mixing of aloe with vegetable glycerin (or glycerol as it is also known) and zinc oxide (micronized in this case) and that we have achieved a superior product using this technique. This would be the so called “aloe-water” phase that would be subsequently mixed at high speed with the so-called “oil-phase”. Blending or homogenizing would be accomplished using either the aloe-water phase or oil phase and in so doing, the aloe would not be necessarily diluted with water until after the full addition and blending of the inorganic sun-block agents. Homogenization may be used to provide SPF values higher than 15. Water dilution during or after blending is acceptable but not necessary and in some cases it may be undesirable. It is also desirable to add additional inorganic sunblocking agents directly to the oil phase to insure SPF values greater than 15. Often, it is necessary to add the inorganic sunblocking agents to both phases (oil and water) to provide a superior formulation.

Eastman NutriLayer™ Phytolipids are natural products derived from rice and intended for use in personal care applications. Phytolipids are a concentrate of phytosterols, tocopherols, tocotrienols, squalene, and rice bran waxes.

Both saturated and unsaturated phytosterols are present in NutriLayer™. The phytosterols present at higher concentration include campesterol, β-sitosterol acetate, brassi-casterol, α-sitosterol, β-sitosterol, and cycloartenol.

NutriLayer™ contains low levels of oleic, linoleic, and stearic acids. Other acids typically found in rice bran wax, such as palmitic, behenic, and lignoceric acids have not been specifically identified in NutriLayer™ but may be present at low levels.

The aloe and specifically single species of aloe as described above, seems particularly well-suited (with and without the use of glycerin) to provide an emulsion that is homogeneous and can achieve SPF values in the range of 15 or greater using 14% or more (by weight) of the inorganic sun-block agents. Ratios of 4 or 3.5:1 or 3.3:1 (weight percent) of ZnO to titanium dioxide (both in the micronized version) have been used to achieve SPF 30 or more. Micronized sun-block agents are best for this emulsion as they provide the best surface area-volume ratio for proper wetting of the ZnO and other micronized inorganic/organic particles. Proper wetting is more readily achieved and results in long term stability when homogenization is employed.

The well known and commercially available “SPF boosters” have almost without exception proven to be toxic such that the present disclosure provides for the use of only SPF boosters that are not toxic. The use of phospholipids or liposomes described above may also provided the required oil-water dispersion and thus also boost SPF.

Below are listed additional components which have been or could be added to the basic SPF composition to boost immuno-responsiveness, SPF values, and cytoprotectiveness without introducing toxicity (cell killing agents).

It is known that the use of green tea extract may be effective in reducing sunburn. Green tea is a powerful antioxidant that neutralizes free radicals from UV radiation and helps protect skin cells by its photoprotective effect on human skin and its polyphenolic antioxidant contents. Green tea protection works in the cell after exposure to ultraviolet rays. Studies suggest it causes abnormal cells to kill themselves, a type of programmed cell suicide that prevents the development of abnormal growths. Green tea inhibits UVB-induced erythema response in the skin (redness reaction). At the same time it supports the production of melanin, the skin's own natural sunburn protection. Thus green tea helps reduce the risk of sunburn and boosts SPF.

Tocopherol, or Vitamin E oil, is a fat-soluble vitamin in eight forms that is an important antioxidant. Vitamin E is often used in skin creams and lotions because it is believed to play a role in encouraging skin healing and reducing scarring after injuries such as burns. Natural vitamin E exists in eight different forms or isomers, four tocopherols and four tocotrienols. All isomers have a chromanol ring, with a hydroxyl group which can donate a hydrogen atom to reduce free radicals and a hydrophobic side chain which allows for penetration into biological membranes. There is an alpha, beta, gamma and delta form of both the tocopherols and tocotrienols, determined by the number of methyl groups on the chromanol ring. Each form has its own biological activity, the measure of potency or functional use in the body. For the present disclosure, the most stable forms of Vitamin E are desired.

Rosehip, also called the rose haw, is the pomaceous fruit of the rose plant and a powerful antioxidant. It is typically red to orange but may be dark purple to black in some species. Particularly high in Vitamin C, with about 1700-2000 mg per 100 g in the dried product, it is one of the richest plant sources of the vitamin. It also contains vitamins A, D and E, and antioxidant flavonoids. Rosehip can be used as an emollient in the present disclosure. The use of vitamin C (ascorbic acid or other available forms of Vitamin C) in sunscreen or sunblock formulations should be in a stabilized form such as Magnesium ascorbyl phosphate. For the present disclosure and associated formulations, the most stable form of Vitamin C can be incorporated.

Keratins may provide an SPF boost to the present compositions. Keratins are a family of fibrous structural proteins; tough and insoluble, they form the hard but nonmineralized structures found in reptiles, birds and mammals. They are rivaled in biological toughness only by chitin, a cellulose-like polymer of glucosamine and the main constituent of the exoskeletons of arthropods. The properties which make structural proteins like keratins useful depend on their supermolecular aggregation. These depend on the properties of the individual polypeptide strands, which depend in turn on their amino acid composition and sequence. The α-helix and β-sheet motifs, and disulfide bridges, are crucial to the conformations of globular, functional proteins like enzymes, many of which operate semi-independently, but they take on a completely dominant role in the architecture and aggregation of keratins. Keratins contain a high proportion of the smallest of the 20 amino acids, glycine, whose “side group” is a single hydrogen atom; also the next smallest, alanine, with a small and uncharged methyl group. In the case of P-sheets, this allows sterically-unhindered hydrogen bonding between the amino and carboxyl groups of peptide bonds on adjacent protein chains, facilitating their close alignment and strong binding. Fibrous keratin molecules can twist around each other to form helical intermediate filaments. Sucrose stearate is usually a white or light brown block or powder, with little or no smell and no taste. It is an exceptionally mild emulsifier derived from sugar and coconut or palm oil. Sucrose stearate is made by combining sugar with Stearic Acid. Cane sugar is a sweetening agent and food which can act as a preservative and antioxidant, and stearic acid is a natural fatty acid derived from coconut or palm oil. Because it is made from vegetable sources it is completely biodegradable. One commercially available form of sucrose stearate is Crodesta® F-160, manufactured by Croda of Yorkshire, England.

Lanolin is a thick natural moisturizer to soothe and protect skin. It is derived primarily from the oil glands in sheep's wool, also known as wool oil, wool wax, wool fat, or wool grease. Wool fat is a mixture of many different chemical compounds, including cholesterol and the esters derived from ‘fatty’ acids containing 18 to 26 carbon atoms. Lanolin is used in many skin formulas to prevent possible irritation from other oils. It functions as a salve and an emollient by sealing in your body's moisture, and is a natural water repellant. Lanolin forms an emulsion with water that's easily absorbed by the skin, softening it and preventing it from frying and cracking. It is used for dry skin, sunburn, and windburn, and should also boost SPF. The use of food grade lecithin is another natural product that would enhance the compositions of the present disclosure in a similar manner as described for lanolin and as such is a compound included as an optional ingredient.

A number of oils are used in commercial sunblocks as SPF boosters. Such oils may be effective at boosting SPF on their own in some cases, or in combination (synergistically) with other oils in other cases. Among these oils are sunflower oil, safflower oil, almond oil, rice bran oil, eucalyptus oil, sesame oil, orange oil, jojoba oil, rosemary oil, peru balsam oil, grape seed oil, pomegranate seed oil, broccoli seed oil, macadamian nut oil etc. Certain waxes may also have a positive SPF effect, including beeswax, orange wax, synthetic waxes and the like. Silicone oils are the usual choice and can be used in the composition of the present invention if they are non-toxic and more precisely if they can identified as non-endocrine disrupting agents. Dow Corning silicone fluids 1401, 1501, and 2-1184 have been identified as such and other silicone fluids may exhibit suitable properties as well.

Beeswax is a product from a bee hive. Beeswax is secreted by honeybees of a certain age in the form of thin scales. It is a tough wax formed from a mixture of several compounds; its main components are palmitate, palmitoleate, hydroxypalmitate and oleate esters of long-chain (30-32 carbons) aliphatic alcohols, with the ratio of triacontanylpalmitate CH₃(CH₂)₂₉O-CO-(CH₂)₁₄CH₃ to cerotic acid CH₃(CH₂)₂₄COOH, the two principal components, in a ratio of 6:1. Beeswax is used commercially to make fine candles, cosmetics and pharmaceuticals including bone wax (cosmetics and pharmaceuticals account for 60% of total consumption), in polishing materials (particularly shoe polish), as a component of modeling waxes, and in a variety of other products. For the present disclosure, the use of Hydroxyoctacosanyl hydroxystearate is not preferred but may have to be used as a beeswax substitute as a consistency regulator and emulsion stabilizer due to recent decreases in the bee population in North America. Japan wax is another substitute that may not be used. Beeswax's primary use in the present disclosure is to increase the water-resistant capabilities of the composition. The beeswax can also be impregnated with sun-block materials (micronized zinc oxide and titanium dioxide, etc.) in order to prevent these materials from being easily washed away during use while maintaining SPF values for longer times. High altitude beeswax is preferred as the SPF value is higher for this substance from bees living at elevations well above sea level. The use of bee pollen and propolis as well as flower pollen for the compositions of the present disclosure, has already been documented. Phytolipids are natural products derived from rice and intended for use in personal care applications. Phytolipids are a concentrate of phytosterols, tocopherols, tocotrienols, squalene, and rice bran waxes. These materials can be used for sunblocking agents and to provide smooth and powdery textures also. Bee pollen, flower pollen, and propolis can also be used to aid in SPF boosting and also to help provide a smooth and powdery texture.

### Preservatives

Skin care products do not last forever. Just like food, all natural skin care products will eventually deteriorate. Chemical preservatives are generally used in the industry because they are much cheaper than, and extend the shelf life of the product more than, natural alternatives. The preferred preservative in the present disclosure is Biovert®, a product of Arch Chemicals®. Biovert® is a system of two linked preparations, which by themselves do not offer antimicrobial efficacy, but together offer anti-microbial efficacy. Biovert® mimics a naturally occurring antimicrobial-antioxidant protection system. When the two-part system is combined, a cascade of linked reactions takes place to generate antimicrobial products in situ. The cascade is initiated by the action of the glucose oxidase enzyme in the presence of its substrate (glucose) and oxygen. This generates H₂O₂, which is used by the lactoperoxidase to catalyze the oxidation of I⁻ and SCN⁻ anions, forming hypoiodite and hypothiocyanate which have antimicrobial activity. The result is rapid microbial cell death.

Other natural preservatives include tea tree and thyme essential oils, grapefruit seed extract, and D-alpha Tocopherol Acetate (Vitamin E). Both ZnO and titanium dioxide are anti-bacterial agents and will aid in the ability to preserve the compositions of the present disclosure.

One embodiment of the present disclosure includes; a sunblock composition comprising; components by weight;
(a) between about 5% and 25% micronized zinc dioxide,
(b) between about 0% and 25% micronized titanium oxide,
(c) between about 0% and 25% glass microspheres,
(d) between about and 1% and 20% glycerin and/or aloe
(e) between about 0.05% and 20% of a carrier oil,
(f) between about 0.05% and 10% of an emulsifier selected from the group consisting of fatty acid salts, fatty acid sucrose esters, phospholipids, borate salts, cocoate esters, essential oils, non-petroleum derived waxes, chitosan, glycerine or aloe,
(g) between about 10% and 95% water and/or ethanol
(h) between about 0% and 5% of a thickening agent
(i) between about 0 and 5% of a pigment

The embodiment also includes a method of preparing the composition comprising:
a) mixing any combination of components (a)-(e) and optionally (f) at a sufficient temperature for a sufficient time period and stirring rate to reduce or eliminate any aggregation or clumping of solids resulting in a first homogeneous mixture
b) mixing any combination of components g, h and i, and optionally f at a sufficient temperature, time, and at a sufficient stirring rate to reduce or eliminate any aggregation or clumping of solids resulting in a second homogeneous mixture
c) combining the mixtures from steps a) and b) at a stir rate sufficient to provide a final composition which is transparent, translucent, or matches any desired skin color or skin tone, wherein component (f) is present in at least one of the first homogeneous mixture or the second homogeneous mixture.

In addition, the composition comprises greater than 90% of ingredients that do not kill cells on contact with the cells wherein 90% or greater human ovarian carcinoma cells remain alive after exposure to the composition according to the LUMI-CELL™ ER Bioassay, or the composition comprises greater than 95% of ingredients that do not kill cells on contact with said cells wherein 95% or greater human ovarian carcinoma cells remain alive after exposure to said composition according to the LUMI-CELL™ ER Bioassay or the composition comprises greater than 98% of ingredients that do not kill cells on contact with said cells wherein 98% or greater human ovarian carcinoma cells remain alive after exposure to said composition according to the LUMI-CELL™ ER Bioassay or the composition comprises 100% of ingredients that do not kill cells on contact with said cells, wherein 100% of human ovarian carcinoma cells remain alive after exposure to said composition according to the LUMI-CELL™ ER Bioassay.

The composition of this embodiment comprises at least one of the following components; an occlusion providing a layer of oil on the surface of the skin to slow water loss and thus increase the moisture content of the stratum corneum, a humectant increasing the water-holding capacity of the stratum corneum and a lubricant adding slip or glide across the skin.

In a separate embodiment, another sunblock composition consisting essentially of components by weight;
(a) between about 5% and 25% micronized zinc dioxide,
(b) between about 0% and 25% micronized titanium oxide,
(c) between about 0% and 25% glass microspheres,
(d) between about and 1% and 20% glycerin and/or aloe
(e) between about 0.05% and 20% of a carrier oil,
(f) between about 0.05% and 10% of an emulsifier selected from the group consisting of fatty acid salts, fatty acid sucrose esters, phospholipids, borate salts, cocoate esters, essential oils, non-petroleum derived waxes, chitosan, glycerine or aloe,
(g) between about 10% and 95% water and/or ethanol
(h) between about 0% and 5% of a thickening agent
(i) between about 0 and 5% of a pigment

The embodiment also includes a method of preparing the composition consisting essentially of:
a) mixing any combination of components (a)-(e) and optionally (f) at a sufficient temperature for a sufficient time period and stirring rate to reduce or eliminate any aggregation or clumping of solids resulting in a first homogeneous mixture
b) mixing any combination of components g, h and i, and optionally f at a sufficient temperature, time, and at a sufficient stirring rate to reduce or eliminate any aggregation or clumping of solids resulting in a second homogeneous mixture
c) combining the mixtures from steps a) and b) at a stir rate sufficient to provide a final composition which is transparent, translucent, or matches any desired skin color or skin tone, wherein component (f) is present in at least one of the first homogeneous mixture or the second homogeneous mixture.

In addition, the composition consists essentially of greater than 90% of ingredients that do not kill cells on contact with the cells wherein 90% or greater human ovarian carcinoma cells remain alive after exposure to the composition according to the LUMI-CELL™ ER Bioassay, or the composition consists essentially of greater than 95% of ingredients that do not kill cells on contact with said cells wherein 95% or greater human ovarian carcinoma cells remain alive after exposure to said composition according to the LUMI-CELL™ ER Bioassay or the composition consists essentially of greater than 98% of ingredients that do not kill cells on contact with said cells wherein 98% or greater human ovarian carcinoma cells remain alive after exposure to said composition according to the LUMI-CELL™ ER Bioassay or the composition consists essentially of 100% of ingredients that do not kill cells on contact with said cells, wherein 100% of human ovarian carcinoma cells remain alive after exposure to said composition according to the LUMI-CELL™ ER Bioassay.

The composition of this embodiment comprises at least one of the following components; an occlusion providing a layer of oil on the surface of the skin to slow water loss and thus increase the moisture content of the stratum corneum, a humectant increasing the water-holding capacity of the stratum corneum and a lubricant adding slip or glide across the skin.

### Methods of Making the Formulations

One possible method which is presented as an example of producing formulations for composing the sunblock composition of the present disclosure, may be performed using a two-vessel method, in which the oil and aloe or water or aloe/water phases are individually prepared. This process produces a smooth, uniform, white to light ivory emulsion, especially in conjunction with the use of an homogenizer, in that it is satisfactory when the inorganic particles are sufficiently dispersed to provide desired SPF values. When combined with ultramarine pigments, including iron oxides, color will change and may also provide a clear or at least matching appearance (using the micronized inorganics) as the composition is applied to the skin. In accordance with a two-vessel process, an aloe or water or aloe/water phase can be prepared by measuring deionized water into a receptacle and mixing. Xanthan gum is sprinkled and mixed until free from lumps. Carrageenan is then mixed in until freed from lumps. (Carrageenan could serve as an alternative to xanthan gum throughout the present disclosure.) The mixture is optionally slowly heated to approximately 80° C., although room temperature or below is preferred. Vegetable glycerin is then added, followed by aloe vera gel. The composite is mixed until completely uniform. The inorganic sunblocking additives such as zinc oxide and titanium dioxide as well as silicon dioxide, the pigments, and glass microspheres can all be added to this phase as well as to the oil phase described below.

The oil phase can be prepared separately in another vessel, at approximately 75° C. Sun-block agents comprising the following are mixed together (with an homogenizer or high speed mixer) until dissolved: refined sunflower oil, lanolin, phospholipids or liposomes, coconut oil, stearic acid, beta carotene, orange wax, beeswax, essential oils, and Vitamin E oil. When mixed, sucrose stearate is slowly added. While maintaining a temperature of 75° C., micronized zinc oxide is sprinkled very slowly and homogenized until smooth and uniform. Cosmetic microspheres and/or titanium dioxide can then be added in the same fashion as the micronized zinc oxide. The temperature of the mixture is raised to 80° C., and the water phase as described above is then added to the oil phase under heavy mixing conditions. Mixing should continue for at least 30 minutes until the mixture is smooth and homogenous. It is preferable to perform the mixing at room temperature or below.

The combined mixture can then be cooled to 45° C. or below. The following ingredients can then be individually added, mixing each well before adding another: aloe vera gel, granular borax, grapefruit seed extract, ascorbyl palmitate, butter milk powder, milk powder. Preservatives are then added and can comprise the following: Biovert® substrate, glucose, lactoperoxidase, and glucose oxidase. Essential oils, for example rosemary oil, peru balsam oil, and olibanum oil (frankincense) are then added to provide fragrance and mixed preferably with homogenization until smooth and homogenous.

An alternative method for formulating the composition is as follows: the formulation is prepared using a two-vessel method, in which the oil and aloe or water or aloe/water phases are individually prepared. In accordance with this two-vessel process, an aloe or water or aloe/water phase is prepared by measuring deionized water and/or aloe into a receptacle and mixing with an homogenizer as it the preferred method through this disclosure. Carrageenan is then mixed in until freed from lumps. The mixture is optionally slowly heated to approximately 80° C., although room temperature or below is preferred. Vegetable glycerin is then added, followed by aloe vera gel. The composite is mixed via an homogenizer until completely uniform. The inorganic sunblocking additives such as zinc oxide and titanium dioxide as well as silicon dioxide, the pigments, and glass microspheres can all be added to this phase as well as to the oil phase described below.

The oil phase is prepared separately in another vessel, at approximately 75° C. Sun-block agents comprising the following are mixed together until dissolved (preferentially with an homogenizer): jojoba oil, rice bran oil, lanolin, phospholipids or liposomes, stearic acid, orange wax, beeswax, essential oils, and Vitamin E oil. While maintaining a temperature of 75° C., micronized zinc oxide and titanium dioxide are sprinkled very slowly and homogenized until smooth and uniform. The temperature of the mixture is raised to 80° C., and the water phase as described above is then added to the oil phase under heavy mixing conditions. Homogenization mixing should continue for at least 30 minutes until the mixture is smooth and homogenous. Based on the recent decreases in the North American bee population, beeswax may become impossible to obtain or too expensive to purchase. In today's market, there is a range of waxes based on plants (vegetables or fruits) that are non-paraffinic. It may be necessary to substitute for the beeswax with a plant wax such as bayberry wax, candelilla wax, carnauba wax, castor wax, esparto wax, Japan wax (from the berries of Rhus and Toxicodendron), ouricury wax, or rice bran wax. Each of these waxes can be impregnated with or mixed with zinc oxide or any of the other sunblocking additives already provided for and described in this disclosure.

The combined mixture can then be cooled to 45° C. Again, it is preferable to conduct the mixing at room temperature or below. The following ingredients can then be individually added, mixing each well before adding another: aloe vera gel, grapefruit seed extract, ascorbyl palmitate. Preservatives are then added and can comprise the following: Biovert Substrate®, glucose, lactoperoxidase, and glucose oxidase. Essential oils, for example rosemary oil, peru balsam oil, and olibanum oil (frankincense) are then added to provide fragrance and mixed until smooth and homogenous.

The following examples serve as illustrations of the compositions of the present disclosure, however, they do not limit the scope of the disclosure described herein. Also, the examples below indicate the exact amounts of each constituent to facilitate the preparation of a 1000 gm of the composition. For each constituent, a range of weights and therefore weight percentages can be added to provide a corresponding formulation with similar properties or identical properties including the ability to change viscosity and consistency of the final composition. Ranges are presented in the corresponding claims.

### EXAMPLE I

211.79 ml of deionized water was added to the receptacle. 0.25 grams of xanthan gum was then added to the receptacle. The composition was mixed until free from lumps. 1.0 g of Carrageenan was added to the receptacle. The composition was mixed until free from lumps. The mixture was heated to 80° C. 15.0 grams of vegetable glycerin was then added to the receptacle, along with 15.0 g of Aloe Vera Gel. The ingredients in the receptacle were then mixed until completely uniform. In a second receptacle, 15.0 g of sunflower oil, 30.0 g of phosphatidyl choline, 1.0 g of coconut oil, 80.0 g of carrier oils, 10.0 g of stearic acid, 0.0005 g of beta carotene, 1.0 g of orange wax, 5.0 g of beeswax, and 0.5 g of vitamin E oil (tocopherol) were mixed until all solids were dissolved, and the mixture was heated to 75° C. 5.0 g Crodesta F-160® (produced by Croda USA) was slowly added, while maintaining the temperature at 75° C. 80.0 grams of micronized zinc oxide (Z-Cote®) was sprinkled in slowly and homogenized until smooth and uniform. 10.0 grams of Sensient Cosmetic Microspheres CM-111® was sprinkled in slowly and homogenized until smooth and uniform. The temperature of this receptacle was increased to 80° C. The first receptacle was then added to the second with vigorous mixing at 80° C. Mixing (preferably by an homogenizer) continued for 30 minutes until the composite was smooth and homogenous. The temperature of the receptacle was lowered to 45° C. 5.0 grams Aloe Vera gel was added, while mixing thoroughly. 0.2 g of borax granular was added, while mixing thoroughly. 5.0 grams of Grapefruit Seed Extract (GSE) was added, while mixing thoroughly. 0.5 grams of ascorbyl palmitate was added, while mixing thoroughly. 0.5 grams of milk powder and 0.5 grams of buttermilk powder were added, while mixing thoroughly. 5.25 grams of Biovert® substrate, a product of Arch Chemicals and a composite of glucose, lactoperoxidase, and glucose oxidase was then added, mixing thoroughly. 1.0 grams of rosemary oil, 0.5 g of peru balsam oil, and 1 gram of olibanum oil (frankincense) were then added. The receptacle was mixed until smooth and homogenous.

SPF (sun protection factor) can be measured as the ratio of the optical signal through the substrate without sunscreen divided by the optical signal through the substrate coated with the sunscreen. The system is calibrated against a series of sunscreens of known SPF (4 through 36) determined in-vivo using the FDA monograph method (Federal Register, Aug. 25, 1978, Sunscreen drug products for over-the-counter human drugs. pp 38206-38269.) The resulting SPF of the composition of Example I above when measured in-vitro was 31.5 and the composition was aesthetically satisfactory and stable.

### EXAMPLE II

139 ml of deionized water was added to the receptacle. 0.91 g of Carrageenan was added to the receptacle. The composition was mixed until free from lumps. The mixture was heated to 80° C. 13.59 grams of vegetable glycerin was then added to the receptacle. The ingredients in the receptacle were then mixed until completely uniform. In a second receptacle, 22.6 g of rice bran oil, 27.18 g of phosphatidyl choline, 36.24 g of carrier oils, 9.06 g of stearic acid, 0.46 g of orange wax, 11.3 g of beeswax, and 0.91 g of vitamin e oil (tocopherol) were mixed until all solids were dissolved, and the mixture was heated to 75° C. 54.41 grams of micronized zinc oxide (Z-Cote®) was sprinkled in slowly and homogenized until smooth and uniform. 21.85 grams of micronized titanium dioxide was sprinkled in slowly and homogenized until smooth and uniform. The temperature of this receptacle was increased to 80° C. The first receptacle was then added to the second with vigorous mixing at 80° C. Mixing continued for 30 minutes until the composite was smooth and homogenous. The temperature of the receptacle was lowered to 45° C. 1.13 grams of Grapefruit Seed Extract (GSE) was added, while mixing thoroughly. 0.453 grams of ascorbyl palmitate was added, while mixing thoroughly. 0.226 grams of Biovert® substrate, a product of Arch Chemicals and a composite of glucose, lactoperoxidase, and glucose oxidase was then added, mixing thoroughly. 0.5 grams of orange oil was added. The receptacle was mixed until smooth and homogenous.

The resulting SPF of the composition of Example II above when measured in vitro was 30.7 and the composition was aesthetically satisfactory and stable.

### EXAMPLE III

139 ml of deionized water was added to the receptacle. 0.91 g of Carrageenan was added to the receptacle. The composition was mixed until free from lumps. The mixture was heated to 80° C. 13.59 grams of vegetable glycerin and 68.0 grams of Aloe Vera gel were then added to the receptacle. The ingredients in the receptacle were then mixed until completely uniform. In a second receptacle, 22.6 g of rice bran oil, 27.18 g of phosphatidyl choline, 36.24 g of carrier oils, 9.06 g of stearic acid, 0.46 g of orange wax, 11.3 g of beeswax, and 0.91 g of vitamin E oil (tocopherol) were mixed until all solids were dissolved, and the mixture was heated to 75° C. 54.41 grams of micronized zinc oxide (Z-Cote®) was sprinkled in slowly and homogenized until smooth and uniform. 21.85 grams of micronized titanium dioxide was sprinkled in slowly and homogenized until smooth and uniform. The temperature of this receptacle was increased to 80° C. The first receptacle was then added to the second with vigorous mixing at 80° C. Mixing continued for 30 minutes until the composite was smooth and homogenous. The temperature of the receptacle was lowered to 45° C. 22.67 grams of Aloe Vera gel was added, while mixing thoroughly. 1.13 grams of Grapefruit Seed Extract (GSE) was added, while mixing thoroughly. 0.453 grams of ascorbyl palmitate was added, while mixing thoroughly. 0.226 grams of Biovert ® substrate, a product of Arch Chemicals and a composite of glucose, lactoperoxidase, and glucose oxidase was then added, mixing thoroughly. 0.5 grams of orange oil was added. The receptacle was mixed until smooth and homogenous.

The resulting SPF of the composition of Example III was measured in vitro to be 30.9 and the composition was aesthetically satisfactory and stable.

### EXAMPLE IV

This example was made in accordance with the method of Example III above, with jojoba oil replacing the rice bran oil in equal quantity by weight. The resulting SPF of the composition of Example IV was tested in vitro to be 30.7 and the composition was aesthetically satisfactory and stable.

### EXAMPLE V

This example was made in accordance with the method of Example IV above, without the addition of micronized titanium dioxide in the composition. The resulting SPF of the composition of Example V was tested in vitro to be 19.6 and the composition was aesthetically satisfactory and stable.

### EXAMPLE VI

This example was made in accordance with the method of Example I above, with twice as much aloe vera gel (a total of 12 g) used in each instance of its addition into the composition. The resulting SPF of the composition of Example VI is in the range of 31.5-33 when measured in vitro and the composition is aesthetically satisfactory.

### EXAMPLE VII

This example was made in accordance with the method of Example I above, with three times as much aloe vera gel (a total of 18 g) used in each instance of its addition into the composition. The resulting SPF of the composition of Example VII is in the range of 31.5-35 when measured in vitro and the composition is aesthetically satisfactory.

### EXAMPLE VIII

This example was made in accordance with the method of Example I above, with four times as much aloe vera gel (a total of 24 g) used in each instance of its addition into the composition. The resulting SPF of the composition of Example VIII is in the range of 31.5-37 when measured in vitro and the composition is aesthetically satisfactory.

### EXAMPLE IX

26.30 ml of deionized water was added to a receptacle. 20.0 grams of Cold Pressed Aloe, 1.75 grams of vegetable glycerin, and 0.25 grams of grapefruit seed extract were mixed into the water. 0.35 g of Xanthan gum was added to the receptacle, with good mixing, until all ingredients were dissolved. The mixture was heated to 40° C. In a second receptacle, 19.2 g of rice bran oil mixed together with 3.5 g of dispersed phosphatidyl choline, 7.0 g of suitable carrier such as castor oil, avocado oil, broccoli seed oil, keratin, micronized or colloidal bentonite, etc. (essential oils or equivalent SPF boosting agents can be used), 0.1 g of orange wax, and 2.5 g of beeswax were mixed until all solids were dissolved, and the mixture was heated to 65° C. When the solution of the second receptacle was heated and became homogenous, 12.0 grams of micronized zinc oxide (Z-Cote®), 4.8 grams of natural source tocopherol (D-alpha), and 4.8 grams of T-Cote® are added to this second receptacle requiring good agitation and maintaining temperature until the micronized powders were properly wetted. A high-energy mixer was used to disperse the ingredients. The first receptacle (water phase) was then added to the second receptacle (oil phase) with high-speed mixing. On a small scale (less than 200 grams), the addition of phases can be reversed. Mixing continued until the composite was cooled. To this mixture, 1.0 gram of Biovert® substrate (a product of Arch Chemicals and a composite of glucose, lactoperoxidase, and glucose oxidase) was then added, mixing thoroughly. 0.05 grams of Biovert® enzyme was added. The receptacle was mixed until smooth and homogenous.

The resulting SPF of the composition of Example IX above when measured in vitro was 30.9 and the composition was aesthetically satisfactory and stable.

### EXAMPLES X-XVIII

Beeswax was heated until melted. Other suitable waxes as described above may be used as substitutes (either individually or in combination). The following ingredients to provide a mixture were then added, in decreasing order of weight: coconut oil, sunflower oil, tocopherol acetate, tocopherol, lanolin, peppermint oil, comfrey root extract, and rosemary extract. No component was added in greater quantity than the initial beeswax. The composition was stirred for several minutes while a constant temperature was maintained above the melting point for beeswax (146 F/62 C). This mixture was then mixed thoroughly preferentially with an homogenizer with each of the above compositions described in Examples I-IX, at a ratio of 90:10 of the current composition to this mixture and then poured into a receptacle and cooled.

| | Current Composition mixed |
|---|---|
| Example # | at 90:10 ratio with Mixture: |
| X | Example 1 |
| XI | Example 2 |
| XII | Example 3 |
| XIII | Example 4 |
| XIV | Example 5 |
| XV | Example 6 |
| XVI | Example 7 |
| XVII | Example 8 |
| XVIII | Example 9 |

### EXAMPLES XIX-XXVII

Beeswax was heated until melted. The following ingredients were then added, in decreasing order of weight: coconut oil, sweet almond oil, tocopheryl acetate, tocopheryl, lanolin, peppermint oil, comfrey root extract, and rosemary extract. Coconut oil was added in greater quantity than the beeswax, all other ingredients in lower quantity. The composition was stirred for several minutes while a constant temperature was maintained above the melting point for beeswax (146 F/62 C). This composition was then mixed thoroughly with the above compositions described in Examples I-IX, at a 90-10 ratio, and then poured into a receptacle and cooled.

| | Current Composition mixed |
|---|---|
| Example # | at a 90-10 ratio with |
| XIX | Example 1 |
| XX | Example 2 |
| XXI | Example 3 |
| XXII | Example 4 |
| XXIII | Example 5 |
| XXIV | Example 6 |
| XXV | Example 7 |
| XXVI | Example 8 |
| XXVII | Example 9 |

### EXAMPLES XXVIII-XXXVI

An oil phase was prepared by combining sunflower oil, stearic acid, coconut oil, beeswax, tocopheryl acetate, orange wax, and beta carotene and stirring the resulting mixture under heat at 80 C until homogeneous. To the oil phase was added sucrose stearate and the resulting mixture was heated, at about 50 C. In a separate container a water phase was prepared by dissolving vegetable glycerin and xanthan gum into deionized water. The water and oil phases were combined. Sodium Borate, Biovert® substrate, and aloe vera gel were added and stirred until homogenous. To the resulting mixture was added fragrance at room temperature and the mixture was allowed to equilibrate overnight. This mixture was then mixed thoroughly preferentially with an homogenizer with each of the above compositions described in Examples IX-XXXVI, at a ratio of 90:10 of the current composition to this mixture and then poured into a receptacle and cooled.

| Example # | Composition+ |
|---|---|
| XXVIII | Example 1 |
| XXIX | Example 2 |
| XXX | Example 3 |
| XXXI | Example 4 |
| XXXII | Example 5 |
| XXXIII | Example 6 |
| XXXIV | Example 7 |
| XXXV | Example 8 |
| XXXVI | Example 9 |

### EXAMPLE XXXVII

423.58 ml of deionized water was added to the receptacle. Alternatively, the use of ethyl alcohol derived from plant matter, is allowed. The water is heated to 80° C., the alcohol to a slightly lower temperature to avoid boiling or rapid evaporation. 15.0 grams of vegetable glycerin was then added to the receptacle, along with 30.0 g of Aloe Vera Gel. The receptacle was then mixed until completely uniform—again an homogenizer is desirable. In a second receptacle, 15.0 g of sunflower oil, 30.0 g phosphatidyl choline, 1.0 g of coconut oil, 80.0 g of carrier oils, 10.0 g of stearic acid, 0.0005 g of beta carotene (30% fs), 1.0 g of orange wax, 1.0 to 5.0 g of beeswax, and 0.5 g of vitamin e oil (tocopherol) were mixed by homogenization if possible, until all solids were dissolved, and the mixture was heated to 75° C. Optionally 5.0 g Crodesta F-160® (produced by Croda USA) was slowly added, while maintaining the temperature at 75° C. 80.0 grams of micronized zinc oxide (Z-Cote®) was sprinkled in slowly and homogenized until smooth and uniform. 10.0 grams of Sensient Cosmetic Microspheres CM-111® was sprinkled in slowly and homogenized until smooth and uniform. The temperature of this receptacle was increased to 80° C. if water and a lower temperature if ethanol is used. The first receptacle was then added to the second with vigorous mixing at 60° C.-80° C.

Mixing continued for 30 minutes until the composite was smooth and homogenous. The temperature of the receptacle was lowered to 45° C. 10.0 grams Aloe Vera gel was added, while mixing thoroughly. 0.2 g of borax granular was added, while mixing thoroughly. 5.0 grams of Grapefruit Seed Extract (GSE) was added, while mixing thoroughly. 0.5 grams of ascorbyl palmitate was added, while mixing thoroughly. 0.5 grams of milk powder and 0.5 grams of buttermilk powder were added, while mixing thoroughly. 5.25 grams of Biovert® substrate, a product of Arch Chemicals and a composite of glucose, lactoperoxidase, and glucose oxidase was then added, mixing thoroughly. 1.0 grams of rosemary oil, 0.5 g of peru balsam oil, and 1 gram of olibanum oil (frankincense) were then added. The receptacle was mixed until smooth and homogenous. The composition was cooled and poured into a container allowing for a spray application product.

Representative Formulations to Generalize the Compositions of the Present Disclosure:

### EXAMPLE A

In one embodiment, a composition of approximately 1000 gms or 1 Kg includes:
between 10 and 700 ml of deionized water,
between 1 and 20 grams of xanthan gum or alternatively or together with
between 1 and 20 grams of carageenan,
between 10 and 300 ml of vegetable based glycerin,
between 10 and 300 ml of cold-pressed aloe vera gel,
between 1 and 100 grams of sunflower oil or other equivalent essential or vegetable based oil
between 1 and 20 grams of phosphatidyl choline or cocoate ester,
between 0 and 100 grams of coconut oil,
between 1 and 100 grams of carrier oils, between 0.1 and 50 grams of stearic acid,
between 0.1 and 10 grams of beta carotene,
between 0.1 and 10 grams of orange wax,
between 0.1 and 10 grams of beeswax,
between 0.1 and 5 grams of tocopherol,
between 0.1 and 30 grams of Crodesta F-160®,
between 0.1 and 50 grams of micronized zinc oxide,
between 0.1 and 50 grams of micronized titanium dioxide,
between 0.1 and 50 grams of Sensient Cosmetic Microspheres CM-111®,
between 0.1 and 10 grams of sodium borate (borax),
between 0.01 and 2 grams of grapefruit seed extract,
between 0.01 and 5 grams of ascorbyl palmitate,
between 0.0 and 10 grams of milk powder,
between 0.0 and 10 grams of buttermilk powder,
between 0.01 and 5 grams of Biovert® substrate,
between 0.01 and 5 grams of a composite of glucose, lactoperoxidase, and glucose oxidase added, and mixed thoroughly with the substrate,
between 0 and 5 grams of rosemary oil,
between 0 and 5 grams of peru balsam oil,
between 0 and 5 grams of frankincense,

SPF (sun protection factor) can be measured as the ratio of the optical signal through the substrate without sunscreen divided by the optical signal through the substrate coated with the sunscreen. The system is calibrated against a series of sunscreens of known SPF (4 through 36) determined in-vivo using the FDA monograph method (Federal Register, Aug. 25, 1978, Sunscreen drug products for over-the-counter human drugs. pp 38206-38269.)

### EXAMPLE B

The following ingredients can also be used;

deionized water, Carrageenan, vegetable glycerin, rice bran oil, phosphatidyl choline, carrier oils, stearic acid, orange wax, beeswax, vitamin e oil (tocopherol), micronized zinc oxide (Z-Cote®), micronized titanium dioxide, Grapefruit Seed Extract (GSE), ascorbyl palmitate, Biovert® substrate, orange oil.

### EXAMPLE C

### The Following Ingredients can Also be Used

deionized water, Carrageenan, vegetable glycerin, Aloe Vera gel, rice bran oil, phosphatidyl choline, carrier oils, stearic acid, orange wax, beeswax, vitamin e oil (tocopherol), micronized zinc oxide (Z-Cote®), micronized titanium dioxide, Grapefruit Seed Extract (GSE), ascorbyl palmitate, Biovert® substrate, orange oil.

### EXAMPLE D

This example was made in accordance with the method of Example A above, with jojoba oil replacing the rice bran oil in equal quantity by weight.

### EXAMPLE E

This example was made in accordance with the method of Example A above, without the addition of micronized titanium dioxide in the composition.

### EXAMPLE F

This example was made in accordance with the method of Example A above, with twice as much aloe vera gel (a total of 12 g) used in each instance of its addition into the composition.

### EXAMPLE G

This example was made in accordance with the method of Example A above, with three times as much aloe vera gel (a total of 18 g) used in each instance of its addition into the composition.

### EXAMPLE H

This example was made in accordance with the method of Example A above, with four times as much aloe vera gel (a total of 24 g) used in each instance of its addition into the composition.

### EXAMPLE G

### The Following Ingredients can Also be Used

deionized water, Cold Pressed Aloe, vegetable glycerin, grapefruit seed extract, Xanthan gum, rice bran oil, dispersed phosphatidyl choline, suitable carrier such as castor oil, avocado oil, broccoli seed oil, keratin, micronized or colloidal bentonite, etc. (essential oils or equivalent SPF boosting agents can be used), orange wax, beeswax, micronized zinc oxide (Z-Cote®), natural source tocopherol (D-alpha), T-Cote®, 1.0 gram of Biovert® substrate.

### EXAMPLES H

### Beeswax

Other suitable waxes as described above may be used as substitutes (either individually or in combination). Coconut oil, sunflower oil, tocopherol acetate, tocopherol, lanolin, peppermint oil, comfrey root extract, and rosemary extract are also added components. No component was added in greater quantity than the initial beeswax. The composition was stirred for several minutes while a constant temperature was maintained above the melting point for beeswax (146 F/62 C). This mixture was then mixed thoroughly preferentially with an homogenizer with each of the above compositions described in Examples A-G, at a ratio of 10:90 of the current composition (Examples A-G) to this mixture and then poured into a receptacle and cooled.

| | Current Composition mixed at |
|---|---|
| Example # | 10:90 ratio with Mixtures A-G: |
| H | Example A |
| I | Example B |
| J | Example C |
| K | Example D |
| L | Example E |
| M | Example F |
| N | Example G |
| O | Example H |

### EXAMPLES P-Y

Beeswax was heated until melted. The following ingredients were then added, in decreasing order of weight: coconut oil, sweet almond oil, tocopheryl acetate, tocopheryl, lanolin, peppermint oil, comfrey root extract, and rosemary extract. Coconut oil was added in greater quantity than the beeswax, all other ingredients in lower quantity. The composition was stirred for several minutes while a constant temperature was maintained above the melting point for beeswax (146 F/62 C). This composition was then mixed thoroughly with the above compositions described in Examples A-H, at a 10:90 ratio, and then poured into a receptacle and cooled.

| | Current Composition mixed |
|---|---|
| Example # | at a 10:90 ratio with |
| P | Example A |
| Q | Example B |
| R | Example C |
| S | Example D |
| T | Example E |
| U | Example F |
| V | Example G |
| X | Example H |
| Y | Example I |

### EXAMPLES AA-II

An oil phase was prepared by combining sunflower oil, stearic acid, coconut oil, beeswax, tocopheryl acetate, orange wax, and beta carotene with sucrose stearate and the following; vegetable glycerin, xanthan gum, deionized water, Sodium Borate, Biovert® substrate, aloe vera gel, fragrance.

This oil phase was used for each composition below by mixing thoroughly with the compositions described in Examples A-I, at a proper ratio, and then poured into a receptacle and cooled if necessary.

| Example # | Composition |
|---|---|
| AA | Example A |
| BB | Example B |
| CC | Example C |
| DD | Example D |
| EE | Example E |
| FF | Example F |
| GG | Example G |
| HH | Example H |
| II | Example I |

### EXAMPLE JJ

### Constituents—Primarily Used for Making an SPF 15 or Greater Spray Alternative to Creams. Lotions, Ointments, Pastes, Etc.

deionized water, the use of ethyl alcohol derived from plant matter, vegetable glycerin, Aloe Vera Gel, sunflower oil, phosphatidyl choline, coconut oil, carrier oils, stearic acid, beta carotene, orange wax, beeswax, vitamin e oil (tocopherol), Optionally Crodesta F-160® (produced by Croda USA), Z-Cote®, Sensient Cosmetic Microspheres CM-111®, borax, Grapefruit Seed Extract (GSE), ascorbyl palmitate, milk powder, buttermilk powder, Biovert® substrate, rosemary oil, peru balsam oil, olibanum oil (frankincense)

The following claims serve to describe formulations that complete compositions of this disclosure, however, they are not intended to limit the scope of the disclosure.

1. A sunblock composition comprising;
these components by weight;
(a) between about 5% and 25% micronized zinc dioxide,
(b) between about 0% and 25% micronized titanium oxide,
(c) between about 0% and 25% glass microspheres,
(d) between about and 1% and 20% glycerin and/or aloe
(e) between about 0.05% and 20% of a carrier oil,
(f) between about 0.05% and 10% of an emulsifier selected from the group comprising fatty acid salts, fatty acid sucrose esters, phospholipids, borate salts, cocoate esters, essential oils, non-petroleum derived waxes, chitosan, glycerine or aloe,
(g) between about 10% and 95% water and/or ethanol
(h) between about 0% and 5% of a thickening agent
(i) between about 0 and 5% of a pigment
j) between about 0 and 2% of a carotenoid.

2. The composition of claim 1, wherein said micronized zinc oxide and micronized titanium dioxide is surface coated to promote wetting of the surface of said micronized zinc oxide or said micronized titanium dioxide.

3. The composition of claim 1, wherein said aloe is cold-pressed aloe gel or liquid or whole leaf aloe gel.

4. The composition of claim 1, wherein said carrier oil is a vegetable oil or essential oil.

5. The composition of claim 1, further comprising an SPF-boosting or SPF-booster agent.

6. The SPF-boosting agent or booster agent of claim 5, wherein said agent is non-toxic such that less than 5% by volume or weight of any toxic substance exists within said booster agent as measured using the LUMI-CELL™ ER Bioassay, wherein 95% or greater human ovarian carcinoma cells remain alive after exposure to said SPF booster agent.

7. The composition of claim 1, further comprising one or more of the following additives in any combination; a silicone oil, antioxidants, free radical scavengers, or anti-inflammatory agents, an inorganic clay, a wood powder, keratin, bee pollen, flower pollen, propolis, beeswax, extracts of cucumber, chamomile, eyebright, pycnogenol, marine collagen, flax oil, witch hazel, omega-3 fatty acids, beta-carotene, beta glucan, vitamin C in any form, vitamin E in any form, vitamin A in any form, any of the B vitamins, arnica, almond oil, primrose oil, astragalus, sasparilla, ligustrum, echinacea, dandelion leaf, royal jelly, lecithin, lanolin or phosphotidyl choline.

8. The composition of claim 1, wherein one or more of any of the amino acids are added.

9. The composition of claim 1, further comprising a naturally occurring or naturally derived fragrance.

10. The composition of claim 9, wherein said fragrance is aroma therapeutic such that said fragrance is also immuno-enhancing.

11. The composition of claim 1, further comprising one or more vitamins, provitamins and/or minerals.

12. The composition of claim 1, further comprising milk powder and/or buttermilk powder.

13. The composition of claim 1, further comprising a preservative.

14. The composition of claim 11, wherein the preservative is Biovert® enzyme, Biovert® substrate, glucose lactoperoxidase, glucose oxidase, tea tree oil, thyme oil, grapefruit seed extract, tocopherol acetate, zinc oxide, titanium dioxide, or any mixture thereof.

15. A method of preparing the composition of claim 1, comprising:
a) mixing any combination of components (a)-(e) and optionally (f) at a sufficient temperature for a sufficient time period and stirring rate to reduce or eliminate any aggregation or clumping of solids resulting in a first homogeneous mixture
b) mixing any combination of components g, h, I and j, and optionally f at a sufficient temperature, time, and at a sufficient stirring rate to reduce or eliminate any aggregation or clumping of solids resulting in a second homogeneous mixture
c) combining the mixtures from steps a) and b) at a stir rate sufficient to provide a final composition which is transparent, translucent, or matches any desired skin color or skin tone, wherein component (f) is present in at least one of said first homogeneous mixture or said second homogeneous mixture.

16. The method of claim 15, wherein titanium dioxide and zinc oxide are present in the form of micronized particles and in ratios of from about 1:4 to 1:3.5 respectively such that formulations leave little or no unsightly white residue when applied to the skin.

17. The method of claim 15, wherein one or more of the mixtures are stirred with a homogenizer wherein said homogenizer agitates or stirs at or above 5000 rpm.

18. The composition of claim 1, wherein said composition comprises greater than 90% of ingredients that do not kill cells on contact with said cells wherein 90% or greater human ovarian carcinoma cells remain alive after exposure to said composition according to the LUMI-CELL™ ER Bioassay.

19. The composition of claim 1, wherein said composition comprises 100% of ingredients that do not kill cells on contact with said cells, wherein 100% of human ovarian carcinoma cells remain alive after exposure to said composition according to the LUMI-CELL™ ER Bioassay.

20. The composition of claim 1 wherein said composition includes at least one of the following components; an occlusion providing a layer of oil on the surface of the skin to slow water loss and thus increase the moisture content of the stratum corneum, a humectant increasing the water-holding capacity of the stratum corneum and a lubricant adding slip or glide across the skin.
